(19) United States
(12) Patent Application Publication
(10) Pub. No.: US 2016/0243165 A1
(43) Pub. Date: Aug. 25, 2016

(54) PROCESSING CELL THERAPY PRODUCTS

(71) Applicant: DANA-FARBER CANCER INSTITUTE, Boston, MA (US)

(72) Inventors: Karl STASKO, Westerly, RI (US); Heather GARRITY, Boston, MA (US)

(21) Appl. No.: 15/030,855

(22) PCT Filed: Oct. 21, 2014
(86) PCT No.: PCT/US2014/061526

§ 371 Date: Apr. 20, 2016

Related U.S. Application Data
(60) Provisional application No. 61/893,495, filed on Oct. 21, 2013.

(51) Int. Cl. A61K 35/18; C12N 5/078; A61K 35/17; B01L 3/00; A01N 1/02

(52) U.S. Cl. CPC A61K 35/18; B01L 3/5021; A01N 1/0284; A61K 35/17; C12N 5/0641; C12N 5/0634; B01L 2300/0672; B01L 2300/041; B01L 2200/0647

(57) ABSTRACT

Systems, methods, and apparatus for processing cell therapy products are provided. A closed system can include a container, an expandable seal, a connector, and a needle for aspirating. The container can have an interior configured to hold at least one cellular therapy product. The expandable seal can be coupled to the container and form a barrier between an external environment and the interior of the container. The connector can be coupled to the expandable seal. The needle can be operatively coupled to the connector, and a distal end of the needle can extend from the connector into the interior of the container. The position of the distal end of the needle can be selectively adjusted between a first depth in the interior of the container and a second depth in the interior of the container without exposing the needle to the external environment.

### CROSS REFERENCE TO RELATED APPLICATIONS

The application claims benefit under 35 U.S.C. §119(e) of U.S. Provisional Application Ser. No. 61/893,495 filed Oct. 21, 2013, the contents of which are herein incorporated by reference in their entirety.

### TECHNICAL FIELD

The present disclosure relates generally to systems, methods, and apparatus for processing cell therapy products. More specifically, the present disclosure relates to a closed system for the processing of cell therapy products, such as blood, bone marrow, and stem cells.

### BACKGROUND

Cell therapy includes therapies in which cellular material is injected into a recipient subject. Cell therapy may be used to reverse, minimize, and/or treat diseases and cellular damage by replacing cellular material, repairing damage and/or stimulating the recipient subject's own immune system. The cellular material may include live or freeze-dried cells or parts of cells, and may be sourced from the recipient subject or from a healthy donor subject. The healthy donor subject may or may not be of the same species. For example, cellular material from an organ, fetus, or embryo of an animal, such as a sheep or cow, may be injected into a human subject. The cellular material is usually processed before being injected into a recipient subject.

A blood transfusion is one form of cell therapy that may be used to, for example, replace lost components of a subject's blood. Whole blood can be collected and then separated by centrifugation into blood components, including red blood cells, plasma, platelets, albumin protein, clotting factor concentrates, cryoprecipitate, fibrinogen concentrate, and immunoglobulins (i.e., antibodies). In addition to testing and other processing steps, one or more components may be isolated. For example, in a luekoreduced blood transfusion procedure, white blood cells (or buffy coat) are removed by filtration to prevent and/or minimize HLA alloimmunization, infections, and other adverse reactions. Following leukoreduction, the remaining components of the blood may be provided intravenously to a recipient subject.

Whole blood or blood components are usually collected and stored in plastic transfusion bags. Multiple transfusion bags may be required in procedures like the luekoreduced blood transfusion for different blood components or due to bag damage or processes intended to prevent bag damage. In addition to cost, the use of multiple transfusion bags may increase the risk of contamination, trauma to cellular components, and the residual accumulation and subsequent loss of components in the bags following transfer.

### SUMMARY

The present disclosure provides systems, methods, and apparatus for processing cell therapy products. In an aspect, a system can include a container, an expandable seal, a connector, and a needle for aspirating. The container can include a top. The expandable seal can be fixed to the container top and form a barrier between an external environment and an interior of the container. The connector can be coupled to the expandable seal. The needle can be operatively coupled to the connector and can extend into the interior of the container. The needle can be movable between a first depth in the container and a second depth in the container without exposing the needle to the external environment.

In one embodiment, a system includes a container with an interior configured to hold at least one cellular therapy product, an expandable seal coupled to the container and forming a barrier between an external environment and the interior of the container, a connector coupled to the expandable seal, and a needle operatively coupled to the connector. The distal end of the needle can extend from the connector into the interior of the container. The position of the distal end of the needle can be selectively adjustable between a first depth in the interior of the container and a second depth in the interior of the container without exposing the needle to the external environment. The needle can be configured for aspirating one or more of the at least one cellular therapy product.

One or more of the following features also can be included. For example, the system can be configured for containing cellular therapy products during centrifugation. The container can be comprised of plastic resistant to puncture by the needle when the needle is moved within the container, for example, when the needle is repositioned within the interior of the container and/or used to aspirate a cellular therapy product from the interior of the container. One or more support elements can be included to support at least a distal portion of the needle, for example, during centrifugation. The expandable seal can include an accordion seal. The connector can be a mechanically-closed device. The mechanically-closed device can be configured to automatically self-seal when not being accessed. A plurality of sterile access tubes can be included, as can at least one port between the interior of the container and the external environment. The at least one port can be one or more of an aspirating needle access port, a vent port, and/or an infusion port.

In another aspect, a method can include providing a closed system containing cellular therapy products. An adjustable needle of the closed system can be moved from a first depth to a second depth. One or more cellular therapy products can be aspirated through the adjustable needle. The closed system containing cellular therapy products can include a container, an expandable seal, a connector, and a needle for aspirating. The container can include a top. The expandable seal can be fixed to the container top and form a barrier between an external environment and an interior of the container. The connector can be coupled to the expandable seal. The needle can be operatively coupled to the connector and can extend into the interior of the container. The needle can be movable between a first depth in the container and a second depth in the container without exposing the needle to the external environment.

In one embodiment, a method includes acquiring a closed system with at least one cellular therapy product, selecting the position of the distal end of the needle to correlate with a location of one or more of the at least one cellular therapy product in the interior of the container, and aspirating the one or more of the at least one cellular therapy product through the needle. In an embodiment, acquiring the closed system with the at least one cellular therapy product includes introducing the at least one cellular therapy product into the interior of the container using at least one of the plurality of access tubes. In an embodiment, the method further includes subjecting the closed system with the at least one cellular therapy product to centrifugation. In an embodiment, the method further includes repositioning the distal end of the needle to correlate with the location of the one or more of the at least one cellular therapy product in the interior of the container. In an embodiment, the method further includes distributing the one or more of the at least one cellular therapy product aspirated from the interior of the container into a freeze bag.

The subject matter described herein provides many advantages for the processing of cell therapy products, such as blood, bone marrow, and stem cells. For example, in some embodiments, by providing and using durable systems and apparatus for processing cell therapy products, cell therapy products, which are irreplaceable at time of transplant, are less likely to be damaged or destroyed by debris during centrifugation or inadvertent punctures during sampling and infusion. Additionally, the use of functionally-closed systems and apparatus can reduce the risk of contamination to cell therapy products without the need, in some cases, for costly clean room facilities and maintenance.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Other systems, processes, and features will become apparent to those skilled in the art upon examination of the following drawings and detailed description. It is intended that all such additional systems, processes, and features be included within this description, be within the scope of the present invention, and be protected by the accompanying claims.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 illustrates a closed system for processing cellular therapy products in accordance with some embodiments.

FIG. 2A illustrates a closed system containing blood post-centrifugation with a needle in a down position in accordance with some embodiments.

FIG. 2B illustrates a closed system containing blood post-centrifugation with a needle in a raised position in accordance with some embodiments.

FIG. 3 illustrates a cross-sectional view of a closed system in accordance with some embodiments.

FIG. 4 illustrates another implementation of a closed system for processing cellular therapy products in accordance with some embodiments.

FIG. 5 illustrates a process for performing apheresis cryopreservation of hematopoietic progenitor cells in accordance with some embodiments.

### DETAILED DESCRIPTION

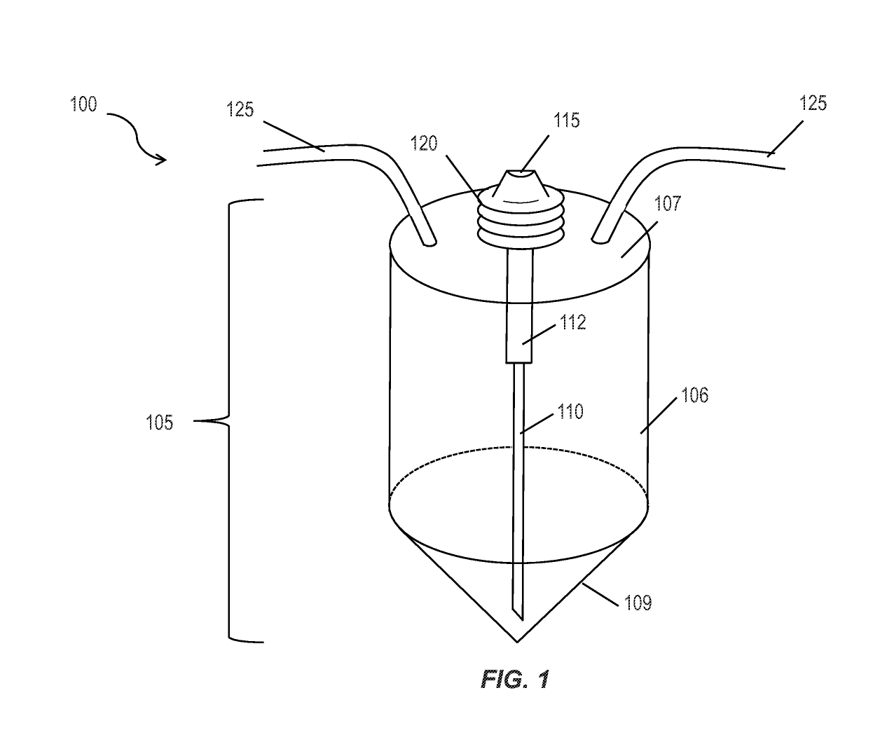

FIG. 1 is a diagram illustrating a system 100 for processing cellular therapy products in accordance with some embodiments. Different embodiments of the system 100 can involve different types of products, different material compositions, and different manufacturing processes. The system can include a container 105, which can be implemented in a wide variety of different shapes, sizes, and configurations. Different embodiments of the container 105 can be used for the storage, transportation, and/or use of a wide variety of cellular therapy products. Cellular therapy products can include live or freeze-dried cells or parts of cells, such as blood components, bone marrow, stem cells, and the like.

Container 105 can be comprised of a durable material, such as polyethylene terephthalate (PET) and/or polypropylene (PP). PET is a thermoplastic polymer resin of the polyester family and can be used in liquid containers, thermoforming applications, and engineering resins, often in combination with glass fiber. PP is also a thermoplastic polymer that can be rugged and resistant to many chemical solvents, bases, and acids. In preferred embodiments, container 105 is resistant to puncture by a needle and has sufficient durability to withstand centrifugal forces applied at a wide range of centrifuge speeds and sample densities. For example, container 105 can have sufficient durability to withstand about 4000 rpm centrifugation in a Beckman JS-4.2 Swinging-Bucket Rotor (4539.5×g) centrifuge (available from Beckman Coulter, Inc. (Brea, Calif.)).

The container 105 includes a body 106, a top portion 107, and a conical bottom portion 109 opposite the top portion 107. In some embodiments, one or both of the top portion 107 and the conical bottom portion 109 can be configured to be detachable/removable from the body 106, for example, as a screw cap. In other embodiments, two or more of the body 106, the top portion 107, and the conical bottom portion 109 are integral to each other and are not configured to be separable from each other. The top portion 107 can be the means by which a container 105 is closed and, in some cases, sealed. The top portion 107 can be in an open position, a closed position, or in some instances, a partially open/partially closed position. The top portion 107 can be comprised of the same material as the body 106.

The shape of container 105 can vary. For example, in FIG. 1 the container 105 is substantially cylindrical in shape. According to some embodiments, the body 106 of the container 105 constitutes the majority of the surface area of the container 105. The body 106 of the container 105 also typically defines the shape, size, and contours of the container 105; however, the length-to-diameter ratio of the container 105 can vary. In some embodiments, the container 105 is a centrifuge tube. The conical bottom portion 109, which can have a more rounded bottom shape, typically serves as the base of the container 105 with the top portion 107 being placed in a vertical position that is higher than most or even all of the body 106, depending on the particular embodiment and application of the container 105.

The size of the container 105 can also vary. For example, the volume capacity of the container 105 can be approximately 50, 250, 500, or 1000 mL. In one embodiment, the container 105 is a cylinder with a volume of about 50 mL, a diameter of about 30 mm, and a height of about 115 mm. In another embodiment, the container 105 has a volume of about 500 mL. In some embodiments, the size and shape of the container 105 is configured to be compatible with one or more standard centrifuges.

The system 100 can include a needle 110 (e.g., an aspirating hypodermic needle with one or more sheaths 112). The needle 110 can be double sheathed, singled sheathed, or unsheathed and can be comprised of metal, plastic, or other suitable material. The distal end of the needle 110 can be blunt. The size of the needle 110 can vary, both in length and in gauge. The diameter of the needle 110 is indicated by the needle gauge. As illustrative examples, needles in common medical use can range from 7 gauge (larger) to 33 gauge (smaller) on the Stubs scale, including 21-gauge needles, which are commonly used for drawing blood for testing purposes, and 16- or 17-gauge needles, which are commonly used for blood donation. In a preferred embodiment of the closed system 100, the needle 110 is 16 gauge.

The needle 110 can be coupled to a connector 115 (e.g., an adapter). The connector 115 can be a needleless closed device, such as an end cap, injection cap, luer-activated device, injection port, and/or mechanical valve. The connector 115 can be adapted to interface with any desired syringe or tubing. In a preferred embodiment, the connector 115 is a mechanically-closed device, which is automatically self-sealing when not accessed, such as a CLAVE® connector (available from ICU Medical, Inc. (San Clemente, Calif.)).

The needle 110 and connector 115 can form a channel for aspiration of particular material residing within the container 105. The needle 110 can have an adjustable position. The needle 110 can be moved to a desired position or level in the container 105; and aspiration can be performed by, for example, coupling the connector 115 to a syringe, pump, or other suction-based device in order to withdraw particular material residing within the container 105 through the needle 110 and connector 115.

The system 100 can further include an expandable seal (or covering) 120. The expandable seal 120 can form a seal with the connector 115 and container 105 to form a barrier between the interior of the container 105 and an external environment. The expandable seal 120 can be, for example, adhered to and/or mechanically coupled to the container 105 and connector 115, and can be composed of a suitable rubber, plastic, or other material. Additionally, the expandable seal 120 can be formed such that its elasticity and/or shape (e.g., similar to an accordion) allows the needle 110 to be manipulated within the container 105. For example, the needle 110 can be lowered and raised within and relative to the container 105. Together, the expandable seal 120, connector 115, and container 105 can form a functionally-closed system such that during use of the system 100, the needle 110 cannot be exposed to the external environment and/or sterility of the system can be improved and/or maintained. Thus, a functionally-closed system can reduce the risk of sample contamination.

Some embodiments of the container 105 include one or more additional openings configured to receive one or more access tubes 125. The access tubes 125 can be included for sterile docking of the system 100, particularly when functionally-closed. For example, the access tubes 125 can provide for introduction of a sample or other product into the container 105. For example, blood can be transferred into the container 105 through a first one of the access tubes 125, while additional solution (e.g., in an apheresis cryopreservation procedure, Plasma-Lyte® A (available from Baxter (Deerfield, Ill.)) may be added to processed blood product) can be transferred into the container 105 through a second and different one of the access tubes 125. Any number of access tubes 125 can be included in the system 100. In one preferred embodiment, six access tubes are included. Additionally, the access tubes 125 can be manufactured with various sizes, lengths, and diameters. In one preferred embodiment, each access tube is about 6 cm in length with an inner diameter of about 1.15 mm and an outer diameter of about 1.6 mm. According to some embodiments, the system 100 includes support elements or features to secure a length of one or more access tubes 125 during centrifugation. For example, clips and/or clasps can be located on and affixed to the container 105 to support a distal portion of one or more access tubes 125.

According to some embodiments, the system 100 can be used for processing cellular therapy products, for example, for blood reduction (e.g., leukoreduction). In the example of reducing blood, blood can be transferred into a closed system 100 through one or more access tubes 125 by sterile docking of the one or more access tubes 125 with a blood supply. The closed system 100 can then be input into a centrifuge. Centrifugation separates the blood into components, including the plasma (supernatant), white cells (buffy coat), and red blood cells (erythrocytes).

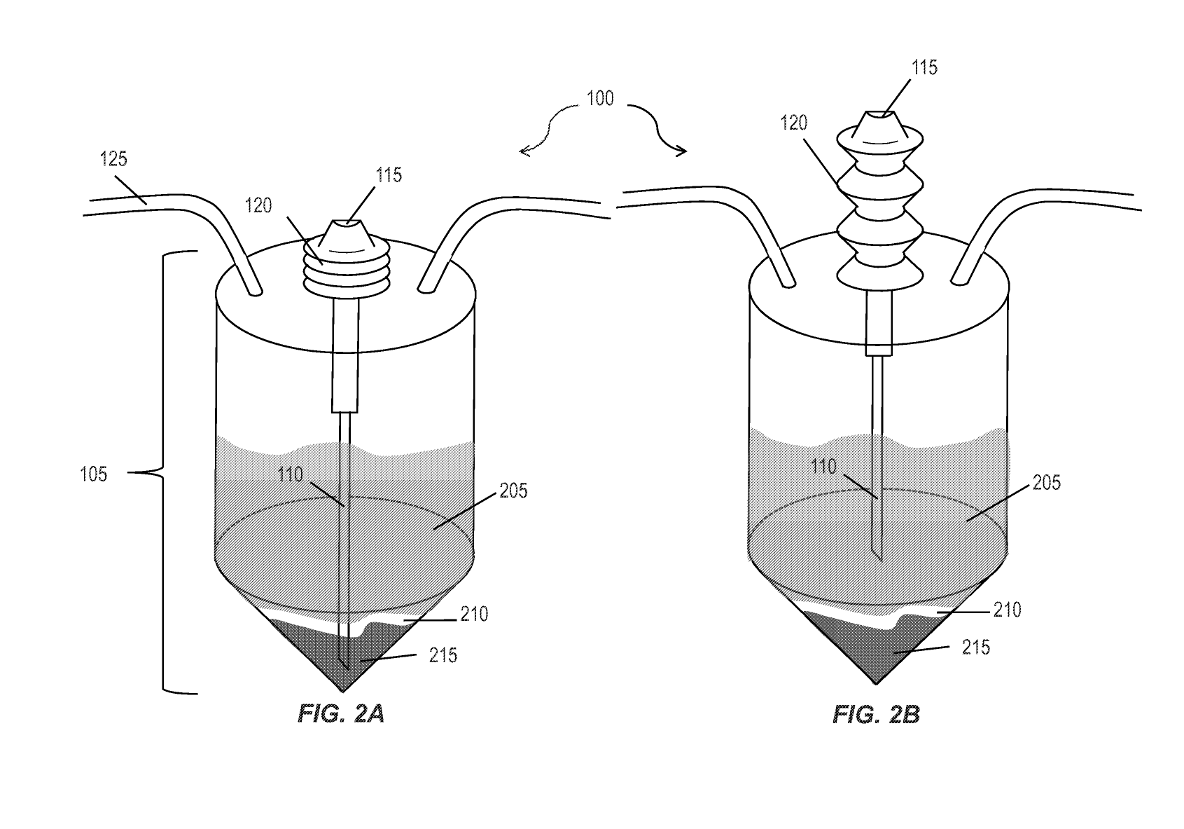

FIG. 2A illustrates a closed system 100 containing post-centrifugation blood with the needle 110 in a down position in accordance with some embodiments. Included within the container 105 are the plasma 205, white blood cells 210, and red blood cells 215. The needle 110 can be manipulated by, for example, pulling the connector 115 and/or expandable seal 120 upwards. FIG. 2B illustrates the closed system 100 with the needle 110 in a raised position. The expandable seal 120 is expanded, allowing the distal end of the needle 110 to be in a different position relative to the container 105. The expansion of the expandable seal 120 allows the distal end of the needle 110 to move in the container 105 without having to remove the needle 110 from the sterile internal environment of the closed system 100. In this manner, the depth by which the needle 110 extends into the container 105 can be controlled. Removal of one or more of the plasma 205, white blood cells 210, and/or red blood cells 215 can be performed by manipulating the needle 110 such that the end of the needle 110 is positioned at a similar level as the material to be removed, and withdrawing the material to be removed through the needle 110 and connector 115, with, for example, a syringe. In the example of leukoreduction, the white blood cells 210 are removed, and the plasma 205 and red blood cells 215 remaining in the container 105 can be used for transfusion.

The vertical range of motion of the needle 110 can vary between implementations. In some embodiments, the vertical range of motion of the needle 110 can be between approximately 25 and 50 percent of the height of the container 105. In other embodiments, the vertical range of motion of the needle 110 is greater than the height of the container 105 (i.e., the distal end of the needle 110 can be adjusted to any desired level within the container 105). Additionally, in some embodiments, one or more of the needle 110, connector 115, and expandable seal 120 can be integral with the container 105. In other embodiments, the needle 110, connector 115, and/or expandable seal 120 can be disconnected (e.g., unfastened) from the container 105.

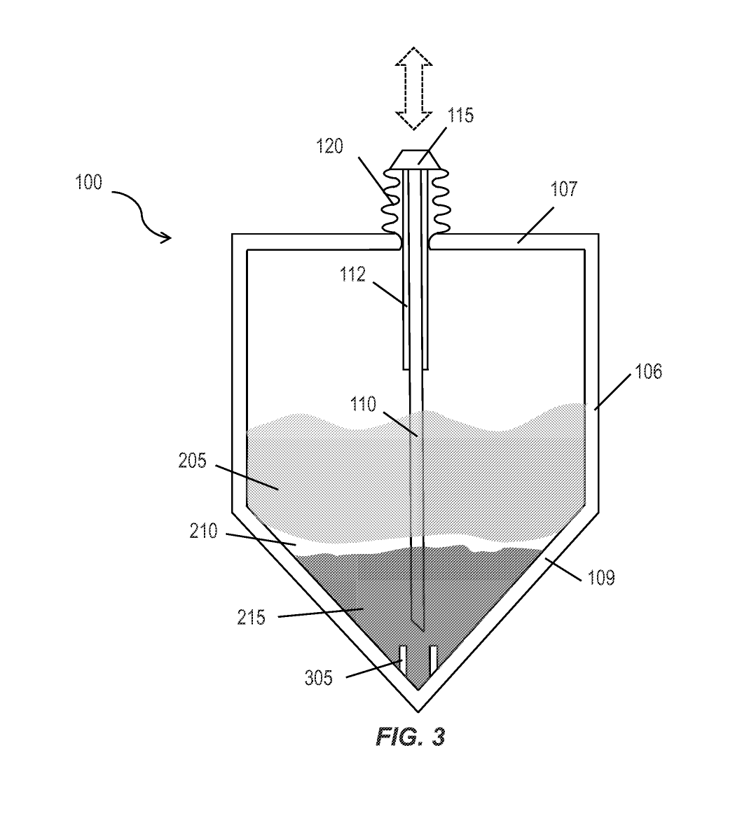

FIG. 3 is a cross-section view of a closed system 100 in accordance with some embodiments. The levels of the plasma 205, white blood cells 210, and red blood cells 215 are evident in FIG. 3. The needle 110 is in a position suitable for removal of the red blood cells 215 from the closed system 100.

Also illustrated in FIG. 3 are support elements or features 305 extending inward from the conical bottom portion 109 of the container body 106. One or more support elements 305 can be configured to support the needle 110 during centrifugation. Centrifugation provides a lateral force on the needle 110. Some centrifuges used to process biological material are spun at maximum angular speeds of 12,000-13,000 rpm, or even greater. One or more support elements 305 can provide support or reinforcement of at least a distal portion of the needle 110 to prevent deformation or significant lateral movement of the needle 110. In the embodiment shown in FIG. 3, prior to centrifugation, the needle 110 can be manipulated such that the distal end of the needle 110 resides within or between the support elements 305. The support elements 305 can, for example, take the form of posts or a cylinder. The support elements 305 can take other forms, for example, as arms or as a disk extending from the body 106, including the conical bottom portion 109, of the container 105 toward the needle 110. The support elements 305 can include one or more apertures, slots, or perforations for allowing passage of material (e.g., blood components) within the container 105.

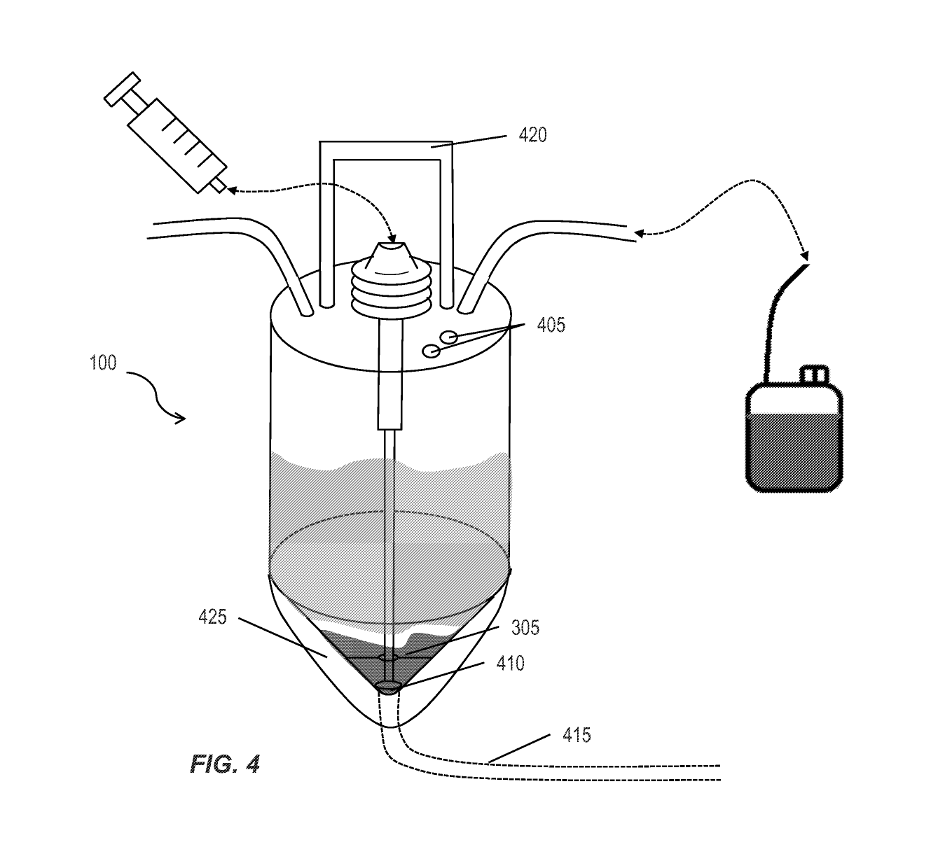

FIG. 4 illustrates another embodiment of a closed system 100 for processing cellular therapy products in accordance with some embodiments. In the embodiment illustrated in FIG. 4, one or more ports 405 can be included on the container 105, for example, on the top portion 107, as shown in FIG. 4. The one or more ports 405 can include a needle access port to allow a second aspirating needle to access the material within the container 105. A second needle access port can be useful, for example, should the needle 110 become disabled. Alternatively or in addition, the one or more ports 405 can include a vent port for stabilizing a pressure differential between the interior of the container 105 and the external environment.

Alternatively or in addition, an infusion port 410 can be included on the container 105, for example, on the conical bottom portion 109, as shown in FIG. 4. The infusion port 410 can include a twist cap or seal that, when removed, allows an infusion line to be attached to the conical bottom 109. The closed system 100 can operate similar to a bedside infusion bag (e.g., an intravenous bag). The closed system 100 can further include an intravenous pole attachment 420 allowing the closed system 100 to be hung on a standard bedside intravenous pole. The closed system 100 can further include a partially or fully removable end cap 425 to protect the infusion port 410 and/or provide a self-standing skirt or structure to the closed system 100. The end cap 425 can connect to the container 105 as, for example, a screw cap, a clip, and the like.

System 100 can include additional aspects. For example, a locking mechanism can be included to secure the connector 115 and/or the expandable seal 120 relative to the container 105 during centrifugation. System 100 can include an integral filter (e.g., about 0.20-0.24 μm membrane) for separating product components. System 100 can be disposable (e.g., intended for a single use) or reusable. Different types of coatings can be used with respect to parts of system 100. For example, the needle 110 and/or the container 105 can include a coating of a silicon gel that separates blood cells from plasma. When blood is centrifuged, the silicone gel forms a layer on top of the buffy coat, allowing the blood plasma to be removed more effectively.

The present disclosure can be applied to many different clinical procedures, including but not limited to the following procedures: cryopreservation; bedside infusion; plasma depletion; red cell reduction; thaw and wash; donor lymphocyte processing; stem cell transplant; hematopoietic transplants; and the like.

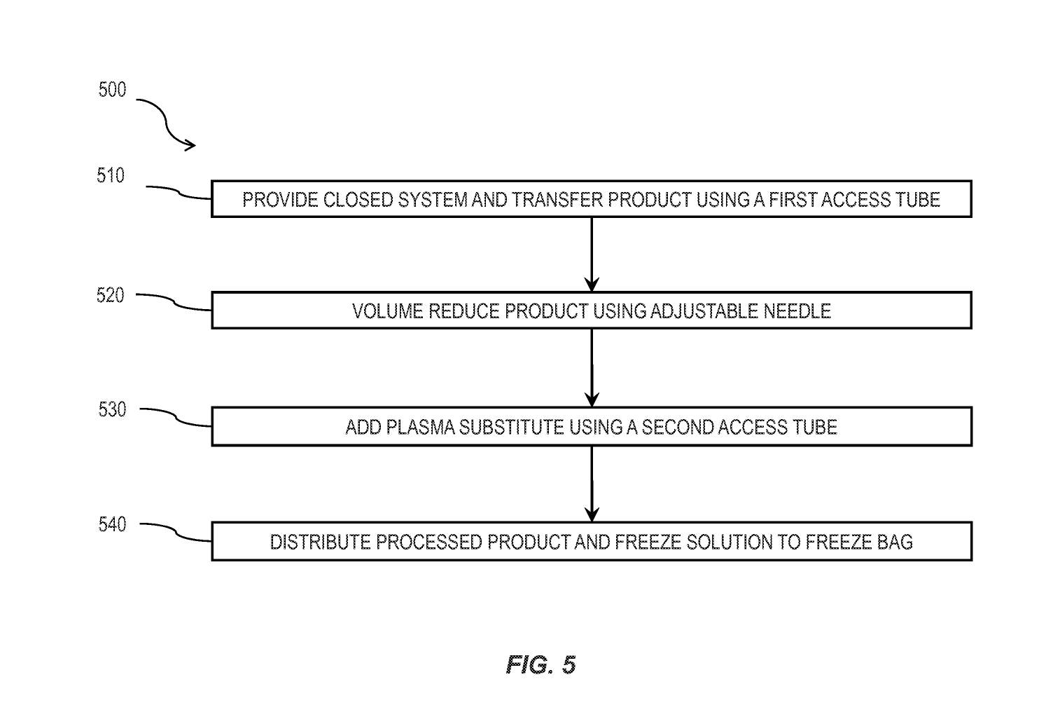

As an example, FIG. 5 is a process flow diagram illustrating a process 500 for performing apheresis cryopreservation of hematopoietic progenitor cells using a closed system 100 in accordance with some embodiments. At step 510, closed system 100 is provided; and cellular matter is transferred into container 105 of the closed system 100 using a first one or more of a plurality of access tubes 125. Following step 510, initial quality control samples can be withdrawn from the container 105 via needle 110; and the entire system 100 with the cellular material can be subjected to centrifugation.

At step 520, the cellular matter is volume reduced by removing the plasma supernatant. The volume reduction can occur by moving the needle 110 of the closed system 100 such that a distal end of the needle 110 is at an appropriate level to withdraw the plasma supernatant. The plasma supernatant may then be withdrawn through the needle 110 and connector 115 (using, e.g., a syringe).

At step 530, a plasma substitute, such as Plasma-Lyte® A (available from Baxter (Deerfield, Ill.)), is added to the container 105 of the closed system 100 using another one or more of the plurality of access tubes 125, different from the first one or more of the plurality of access tube(s) 125 used to introduce the cellular material at step 510. The plasma substitute can be added to increase the cellular therapy product volume to a desired pre-freeze volume. Following step 530, quality control samples can be removed using the needle 110.

At step 540, the closed system 100 and a freezing solution are sterile docked to a harness for distribution of both the freezing solution and the cellular therapy product contained in the closed system 100 into a freeze bag, which can then be frozen.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All publications, patent applications, patents, and other references mentioned herein are incorporated by reference in their entirety.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, the terms “about,” “approximately,” and the like are to be understood to mean+/−10% of the total amount stated, e.g., about 5 would include 4.5 to 5.5, about 10 would include 9 to 11, and about 100 would include 90-110.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the several purposes of the disclosed subject matter. For example, the logic flow depicted in the accompanying figures and described herein does not require the particular order shown, or sequential order, to achieve desirable results. It is important, therefore, that the claims be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter, which is limited only by the claims which follow.

1. A system comprising:
a container with an interior configured to hold at least one cellular therapy product;
an expandable seal coupled to the container and forming a barrier between an external environment and the interior of the container;
a connector coupled to the expandable seal; and
a needle operatively coupled to the connector, a distal end of the needle extending from the connector into the interior of the container, wherein a position of the distal end of the needle is selectively adjustable between a first depth in the interior of the container and a second depth in the interior of the container without exposing the needle to the external environment, the needle being configured for aspirating one or more of the at least one cellular therapy product.

2. The system of claim 1, wherein the system is configured for containing cellular therapy products during centrifugation.

3. The system of claim 1, wherein the container is comprised of a plastic resistant to puncture by the needle when the needle is at least one of repositioned within the interior of the container and used to aspirate a cellular therapy product from the interior of the container.

4. The system of claim 1, further comprising one or more support elements to support at least a distal portion of the needle during centrifugation.

5. The system of claim 1, wherein the expandable seal comprises an accordion seal.

6. The system of claim 1, wherein the connector comprises a mechanically-closed device, the device configured to automatically self-seal when not being accessed.

7. The system of claim 1, further comprising a plurality of sterile access tubes.

8. The system of claim 1, further comprising at least one port between the interior of the container and the external environment, the at least one port being at least one of an aspirating needle access port, a vent port, and an infusion port.

9. A method comprising:
acquiring a closed system with at least one cellular therapy product, the closed system comprising:
a container with the at least one cellular therapy product disposed within an interior of the container;
an expandable seal coupled to the container and forming a barrier between an external environment and the interior of the container;
a connector coupled to the expandable seal; and
a needle operatively coupled to the connector, a distal end of the needle extending from the connector into the interior of the container, wherein a position of the distal end of the needle is selectively adjustable between a first depth in the interior of the container and a second depth in the interior of the container without exposing the needle to the external environment;
selecting the position of the distal end of the needle to correlate with a location of one or more of the at least one cellular therapy product in the interior of the container; and
aspirating the one or more of the at least one cellular therapy product through the needle.

10. The method of claim 9, wherein the closed system is configured for containing cellular therapy products during centrifugation.

11. The method of claim 9, wherein the container is comprised of a plastic resistant to puncture by the needle when the needle is at least one of repositioned within the interior of the container and used to aspirate a cellular therapy product from the interior of the container.

12. The method of claim 9, wherein the closed system further comprises a one or more support elements to support at least a distal portion of the needle during centrifugation.

13. The method of claim 9, wherein the expandable seal comprises an accordion seal.

14. The method of claim 9, wherein the connector comprises a mechanically-closed device, the device configured to automatically self-seal when not being accessed.

15. The method of claim 9, wherein the closed system further comprises a plurality of access tubes.

16. The method of claim 15, wherein acquiring the closed system with the at least one cellular therapy product comprises introducing the at least one cellular therapy product into the interior of the container using at least one of the plurality of access tubes.

17. The method of claim 9, wherein the closed system further comprises at least one port between the interior of the container and the external environment, the at least one port being at least one of an aspirating needle access port, a vent port, and an infusion port.

18. The method of claim 9, further comprising subjecting the closed system with the at least one cellular therapy product to centrifugation.

19. The method of claim 9, further comprising repositioning the distal end of the needle to correlate with the location of the one or more of the at least one cellular therapy product in the interior of the container.

20. The method of claim 9, further comprising distributing the one or more of the at least one cellular therapy product aspirated from the interior of the container into a freeze bag.
